(19) United States
(12) Patent Application Publication
(10) Pub. No.: US 2007/0238164 A1
(43) Pub. Date: Oct. 11, 2007

(54) Plastic microchip for microparticle analysis and method for manufacturing the same

(75) Inventor: Hyun Jin Kim, Yongin-si (KR)

Correspondence Address: FROMMER LAWRENCE & HAUG, 745 FIFTH AVENUE- 10TH FL., NEW YORK, NY, 10151, US

(73) Assignee: SKC CO., LTD.

(21) Appl. No.: 11/732,979

(22) Filed: Apr. 5, 2007

(30) Foreign Application Priority Data
Apr. 10, 2006 (KR) 10-2006-0032494

(51) Int. Cl. C12M 1/34; H01L 21/00

(52) U.S. Cl. 435/287.2; 438/1

(57) ABSTRACT

Disclosed herein is a plastic microchip used in counting the number of microparticles and a method for manufacturing the same and, more particularly, to a plastic microchip including a negative microgrid pattern formed on a lower substrate, a solvent channel and solvent inlets for a solvent welding process, and a method for manufacturing the plastic microchip by injection molding the lower substrate on which a negative microgrid pattern is formed and by injecting a solvent through the solvent inlets so as to fix an upper substrate to the lower substrate.

According to the present invention, it is possible to form a microgrid pattern of a relatively narrow width deeply and uniformly as a negative microgrid pattern is formed on the lower substrate and thereby to provide a clear microgrid pattern, thus facilitating accurate observation of microparticles. Moreover, it is also possible to provide a uniform height of an injection chamber by welding an upper substrate and a lower substrate to each other by a solvent welding process, thus ensuring a more accurate analysis result.

### CROSS-REFERENCE TO RELATED APPLICATION

This application claims the benefit under 35 U.S.C. §119(a) on Korean Patent Application No. 10-2006-0032494 filed on Apr. 10, 2007 the entire contents of which are hereby incorporated by reference.

### BACKGROUND OF THE INVENTION

1. Field of the Invention

The present invention relates to a plastic microchip used in counting or observing microparticles included in a sample of liquid phase and a method for manufacturing the same More particularly, the present invention relates to a plastic microchip including a negative microgrid pattern adapted for counting the number of microparticles and a method for manufacturing the plastic microchip in which a solvent welding process is employed in fixing an upper substrate to a lower substrate.

2. Description of Related Art

In general, microparticles are particles between 1 to 100 μm in size included in a solution and an organic solvent.

Such microparticles may be exemplified by blood cell colonies such as red blood cells, white blood cells, platelets, etc. contained in blood, cell colonies contained in urine, saliva, spinal fluid, etc., yeast colonies in fermented foods such as beer, bacterial colonies and nanoplanktons contained in solution, cells and impurities contained in suspension such as juice, ketchup, milk, etc., mammalian germ cell colonies, impurities contained in incompletely dissolved suspension, various metal and nonmetal crystals contained in solution and solvent, and so on.

Meanwhile, in order to diagnose diseases such as AIDS, leukemia, anemia, etc., to monitor the development of diseases and to examine the therapeutic effects, the number and function of the typical blood cells such as red blood cells, white blood cells, platelets, etc. contained in blood of patients are analyzed.

For example, chronic leukemia can be diagnosed by the number of platelets, kidney diseases, hypoxia, smoking, pulmonary diseases, hemolytic anemia, aplastic anemia, etc. can be diagnosed by the number of red blood cells, and acute typhlitis, leukemia, aplastic anemia, etc. can be diagnosed by the number of white blood cells.

Like this, the measurement of the number of cells such as hemocytes is closely related to the diagnosis of diseases and, especially, the number of the red blood cells is an essential checkout for identifying an anemia and its cause.

Typically, the sizes of the red blood cells are classified into micro, normal, macro and mega and, the analysis results of the size and number of red blood cells can be used as diagnosis data for various diseases as described above.

A healthy man has red blood cells of about 4.4 to 5.6 million/ [symbol] in blood and a healthy woman has red blood cells of about 3.5 to 5 million/ [symbol] in blood.

Meanwhile, a plastic microchip has been widely used in observing and counting the microparticles existing in a liquid phase sample, e.g., the blood cells contained in blood.

The plastic microchip comprises a glass, silicon or plastic substrate including a flow path for injecting a sample containing microparticles, formed by anisotropic etching and having a sample inlet formed on one side thereof and a sample outlet established on the other side thereof.

Microparticles existing in the flow path with appropriate width and height in the plastic microchip can be counted using analysis equipment including an optical microscope, a CCD camera, etc.

Hereinafter, a configuration of a plastic microchip and a method for manufacturing the same in accordance with the conventional art will be described with reference to the accompanying drawings.

FIG. 1 is a perspective view depicting a plastic microchip for measuring the number of blood cells such as red blood cells in accordance with a conventional art, FIG. 2 is a sectional view depicting an upper substrate in the plastic microchip depicted in FIG. 1, FIG. 3 is a sectional view depicting a lower substrate in the plastic microchip depicted in FIG. 1.

As depicted in the figures, the conventional plastic microchip 10 for measuring the number of red blood cells comprises a light transmissive upper substrate 20 including an injection chamber 21 into which a sample is injected, a sample inlet 22 and an outlet 23, and a light transmissive lower substrate 30 including a positive microgrid pattern 31 formed on the top surface thereof. The upper substrate 20 and the lower substrate 30 are welded to each other in an ultrasonic welding process and the like.

Referring to FIG. 2, the upper substrate 20 includes the injection chamber 21, formed in a groove structure having a predetermined depth and providing a space in which a sample is filled, the sample inlet 22, formed penetrating the upper substrate 20 to be connected to one side of the injection chamber 21, through which the sample is injected, and the outlet 23, formed penetrating the upper substrate 20 to be connected to the other side of the injection chamber 21, through which air and an excess of the sample remaining in injection chamber 21 are discharged during the injection of the sample.

If the upper substrate 20 of such a structure stacked on the lower substrate 30 is joined and fixed thereto, the injection chamber 21 formed between the top surface of the lower substrate 30 and the bottom surface of the upper substrate 20 provides a space in which a sample is filled.

Moreover, an indicator 24 indicating a position of the microgrid pattern 31 formed on the lower substrate 30 is included in the top surface of the upper substrate 20.

The upper substrate 20 is made of a light transmissive plastic material such polycarbonate, polymethylmethacrylate, polyethylene, polyethyleneterephthalate and polystyrole by an ordinary injection molding process.

The lower substrate 30 is also formed of a light transmissive plastic material and further includes the microgrid pattern 31, formed on the top surface of the lower substrate 30, for counting the number of cells contained in a sample, as depicted in FIG. 3.

FIGS. 4A to 4D are sectional views illustrating an example of a process of forming a microgrid pattern on the lower substrate to manufacture the plastic microchip depicted in FIG. 1.

A process generally used in the semiconductor process has been applied to a process of forming the positive microgrid pattern 31 on the substrate 30. That is, a photoresist layer is stacked on a plate 32 made of glass, silicon or ceramic by coating photoresist (PR) using a spin coating process and the like. The photoresist layer is patterned through exposure and developing processes to form a mask pattern 33 (FIG. 4A). Subsequently, the plate 32 is etched by using the mask pattern 33 and then the mask pattern 33 is removed to complete a mold 32a on which a negative microgrid pattern is formed (FIG. 4B).

A molten plastic material heated to a predetermined temperature is put into the mold 32a and then cooled on the mold 32a to be hardened (FIG. 4C). Then, the mold 32a is removed (FIG. 4D) to yield a lower substrate 30 on which the positive microgrid pattern 31 is formed.

FIGS. 5A to 5H are sectional views illustrating another example of the process of forming a microgrid pattern on the lower substrate to manufacture the plastic microchip depicted in FIG. 1.

First, a photoresist layer 42 is stacked on a plate 41 made of glass, silicon, ceramic, etc. to be used as a mask by coating photoresist using a spin coating process and the like (FIG. 5A). The photoresist layer 42 is patterned through exposure and developing processes to form a photoresist layer pattern 42a (FIG. 5B).

Subsequently, the plate 41 is etched by using the photoresist layer pattern 42a as an etching mask (FIG. 5C) and then the etching mask is removed through a strip process to prepare a master 41a on which a positive microgrid pattern is formed (FIG. 5D).

Next, as depicted in FIG. 5E, a Ni layer is formed on the master 41a using an electroless plating or electroplating process and then the master 41a is removed to give a mold 43 of a Ni material as depicted in FIG. 5F.

Here, it is possible to surface-treat the master 41a to be charged with electric current using a sputtering, vacuum deposition or electroless plating process just prior to the plating process.

Then, as depicted in FIGS. 5G and 5H, if a molding process is performed using the resulting mold 43, in which a molten plastic material heated to a predetermined temperature is injected into the mold 43 and cooled on the mold 43 to be hardened and then the mold 43 is removed, a lower substrate 30 on which the positive microgrid pattern 31 is formed is manufactured.

Briefly describing a using method of the plastic microchip depicted in FIG. 1, a sample is injected through the sample inlet 22 formed on the upper substrate 20 to fill the injection chamber 21.

Here, air and an excess of the sample remaining in the injection chamber 21 are discharged through the outlet 23 during the injection of the sample. If observing the plastic microchip 10 prepared as described above under an optical microscope, it is possible to measure the number of cells contained in the sample with the microgrid pattern 31 formed on the lower substrate 10.

However, the above-described plastic microchip has several drawbacks in that it requires a lot of time and cost for manufacturing the same since a plastic material is melted and put into a mold to form a lower substrate, thus increasing the manufacture cost. Accordingly, the price of an end product is too high to use the microchip as a disposable.

Moreover, it requires high temperature and high pressure to inject a molten plastic material into a microgrid pattern with a narrow width formed on a mold in order to create a positive microgrid pattern, and further there have been a lot of difficulties in forming a microgrid pattern clearly on the lower substrate.

Furthermore, since the upper substrate and the lower substrate are welded to each other using an ultrasonic welding process, the shape of the upper substrate may be transformed during the welding process, thus making the height of the injection chamber unequal.

The information disclosed in this Background of the invention section is only for enhancement of understanding of the background of the invention and should not be taken as an acknowledgement of any form of suggestion that this information forms the prior art that is already known to a person skilled in the art.

### SUMMARY OF THE INVENTION

Accordingly, the present invention has been made in an effort to solve the above-described drawbacks. In one aspect, the present invention provides a plastic microchip and a method for manufacturing the same that can form a microgrid pattern with a narrow width deeply, clearly and uniformly by forming a negative microgrid pattern on a lower substrate, thus facilitating the observation of sample.

Moreover, in another aspect, the present invention provides a plastic microchip and a method for manufacturing the same that can provide a uniform height of an injection chamber by welding an upper substrate and a lower substrate to each other using a solvent welding process, thus ensuring a more accurate analysis result.

Furthermore, in still another aspect, the present invention provides a plastic microchip and a method for manufacturing the same that can realize a mass production and reduce the cost and time involved therein by injection molding a lower substrate on which a negative microgrid pattern is formed using a stamper for the mass production, compared with the conventional method in which a molten plastic material is put into a mold and cooled on the mold to be cured.

In order to accomplish the above objects, one embodiment of the present invention provides a plastic microchip for microparticle analysis comprising light transmissive upper and lower substrates stacked up and down, an injection chamber defined between the upper and lower substrates, a sample inlet connected to one side of the injection chamber, an outlet connected to the other side of the injection chamber, and a microgrid pattern, formed on the top surface of the lower substrate, for counting the number of microparticles in a sample contained in the injection chamber. The microgrid pattern is formed in a negative structure in such a manner that microlines of a groove shape are arranged in the form of a lattice on the top surface of the lower substrate.

Here, a groove structure is formed adjacent to a wall established along the whole circumference of the injection chamber on the bottom surface of the upper substrate, and the groove structure and the top surface of the lower substrate forms a solvent channel along a predetermined path of the circumference of the injection chamber, and a plurality of solvent inlets is formed penetrating the top surface of the solvent channel to be opened at regular intervals along the solvent channel on the upper substrate.

Meanwhile, one embodiment of the present invention provides a method for manufacturing a plastic microchip for microparticle analysis including light transmissive upper and lower substrates stacked up and down, an injection chamber formed between the upper and lower substrates, a sample inlet connected to one side of the injection chamber, an outlet connected to the other side of the injection chamber, and a microgrid pattern, formed on the top surface of the lower substrate, for counting the number of microparticles in a sample of the injection chamber. The method comprising the steps of:

(a) manufacturing an upper substrate by injection molding a light transmissive plastic;

(b) manufacturing a lower substrate including a negative microgrid pattern formed on the top thereof by injection molding a light transmissive plastic;

(c) surface-treating the upper substrate and the lower substrate; and

(d) welding the upper substrate and the lower substrate to be stacked up and down.

In a preferred embodiment, in step (a), the upper substrate having a groove structure, formed adjacent to a wall provided along the whole circumference of the injection chamber on the bottom surface of the upper substrate, and a plurality of solvent inlets formed penetrating the top to be opened in the groove structure.

In the preferred embodiment, in step (d), the upper substrate and the lower substrate stacked up and down are solvent-welded by injecting a solvent through the respective solvent inlets into a solvent channel formed by the groove structure and the top surface of the lower substrate, the solvent being injected into a boundary between the upper substrate and the lower substrate.

In a further preferred embodiment, step (b) comprises the steps of:

stacking a photoresist layer on a plate;

forming a mask pattern having a negative microgrid pattern on the plate by patterning the photoresist layer through exposure and developing processes;

forming an electrically conductive metal layer on the surface on which the mask pattern is formed;

forming a stamper of a metal material, on which a positive microgrid pattern is formed, on the metal layer by performing an electroless plating or electroplating;

separating the stamper from the mask pattern and washing the stamper separated;

processing the resulting stamper through a series of processes of coating a protective layer, polishing the rear side and cutting to a size capable of being fixed to a mold; and

obtaining a lower substrate on which a negative microgrid pattern is formed by mounting the processed stamper on the mold and then injection molding.

### BRIEF DESCRIPTION OF THE DRAWINGS

The above and other features of the present invention will be described with reference to certain exemplary embodiments thereof illustrated the attached drawings in which:

FIG. 1 is a perspective view depicting a plastic microchip in accordance with a conventional art;

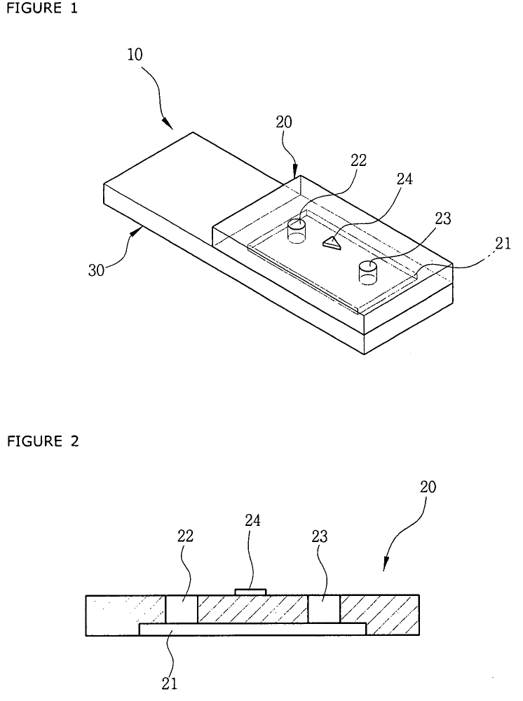

FIG. 2 is a sectional view depicting an upper substrate in the plastic microchip depicted in FIG. 1;

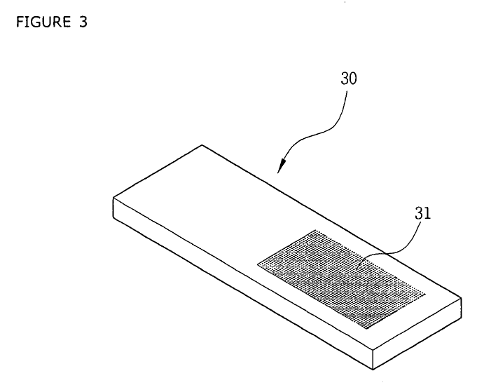

FIG. 3 is a sectional view depicting a lower substrate in the plastic microchip depicted in FIG. 1;

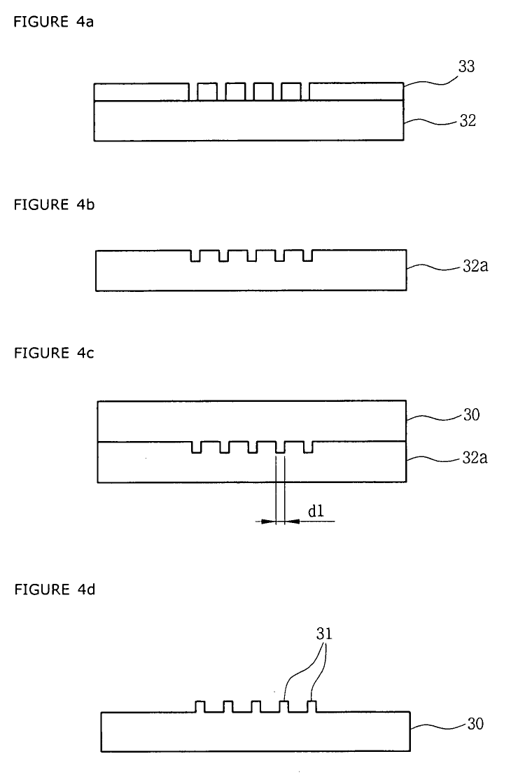

FIGS. 4A to 4D are sectional views illustrating an example of a process of forming a microgrid pattern on the lower substrate to manufacture the plastic microchip depicted in FIG. 1;

FIGS. 5A to 5H are sectional views illustrating another example of a process of forming a microgrid pattern on the lower substrate to manufacture the plastic microchip depicted in FIG. 1;

FIG. 6 is an exploded perspective view of a plastic microchip in accordance with the present invention;

FIGS. 7A and 7B are sectional views of an upper substrate of a plastic microchip in accordance with the present invention, in which FIG. 7A is a sectional view, taken along line A-A of FIG. 6 and FIG. 7B is a sectional view, taken along line B-B of FIG. 6;

FIG. 8 is an enlarged top view of a negative microgrid pattern formed on the lower substrate of the plastic microchip depicted in FIG. 6;

FIGS. 9A to 9G are sectional views illustrating respective processes of manufacturing the lower substrate in accordance with the present invention; and

FIG. 10 is an exploded perspective view depicting another embodiment of the present invention including two injection chambers.

### DETAILED DESCRIPTION OF THE INVENTION

Hereinafter, reference will now be made in detail to the preferred embodiment of the present invention, examples of which are illustrated in the accompanying drawings, wherein like reference numerals refer to like elements throughout.

The present invention relates to a plastic microchip used in counting the number of microparticles contained in a sample of liquid phase such as a solution, an organic solvent, etc. using analysis equipment including an optical microscope, a CCD camera, etc., and a method for manufacturing the same and, more particularly, to a plastic microchip having a negative microgrid pattern for counting the number of microparticles and a method for manufacturing the plastic microchip to which a solvent welding process is applied so as to fix an upper substrate to a lower substrate.

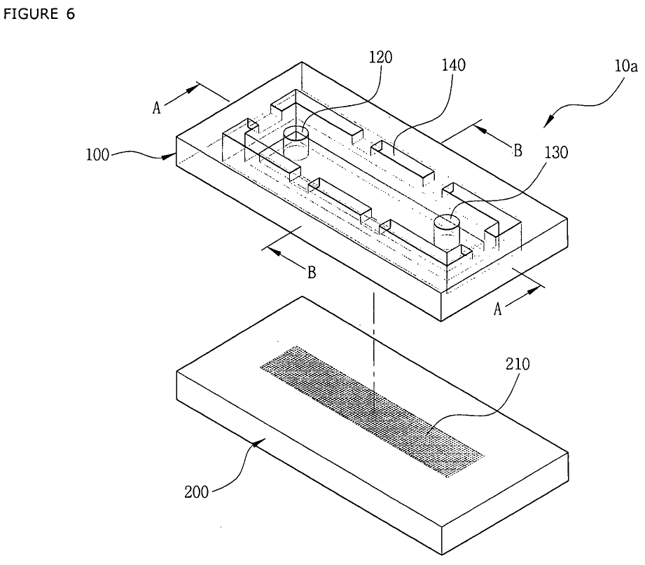

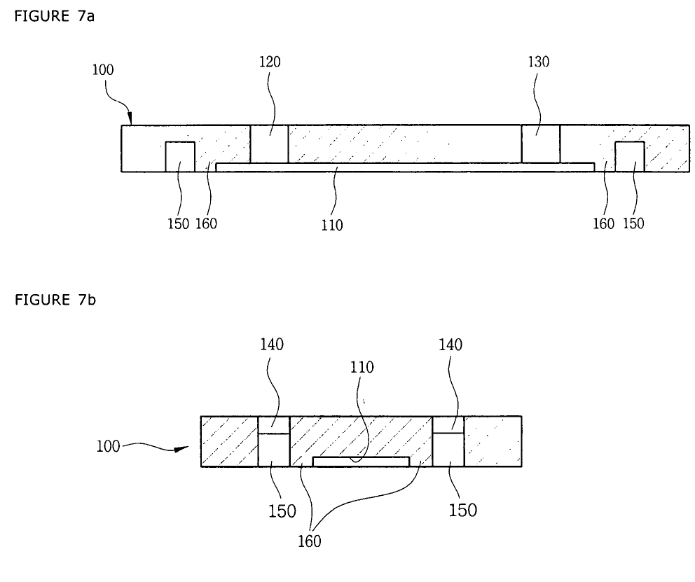

FIG. 6 is an exploded perspective view of a plastic microchip in accordance with the present invention, and FIGS. 7A and 7B are sectional views of an upper substrate of a plastic microchip in accordance with the present invention, in which FIG. 7A is a sectional view, taken along line A-A of FIG. 6 and FIG. 7B is a sectional view, taken along line B-B of FIG. 6.

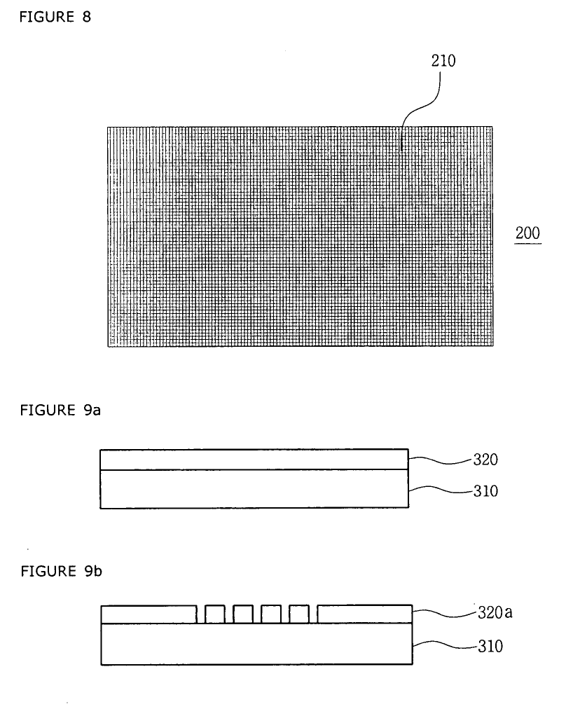

FIG. 8 is an enlarged top view of a negative microgrid pattern formed on the lower substrate of the plastic microchip depicted in FIG. 6.

As described in the figures, the plastic microchip 10a in accordance with the present invention comprises a light transmissive lower substrate 200 on which a negative microgrid pattern 210 for counting the number of microparticles is formed and a light transmissive upper substrate 100 stacked on the lower substrate 200.

Although the upper substrate 100 and the lower substrate 200 separated from each other are depicted in FIG. 6, the plastic microchip 10a of the present invention is provided as an integrated product in which the upper substrate 100 and the lower substrate 200 are stacked and then welded to each other the same as the conventional one.

First, the upper substrate 100 comprises an injection chamber 110 formed in a groove structure of a predetermined depth, a sample inlet 120 formed penetrating the upper substrate 100 to be connected to one side of the injection chamber 110 and an outlet 130 formed penetrating the upper substrate 100 to be connected to the other side of the injection chamber 110.

In a state where the upper substrate 100 and the lower substrate 200 are joined to each other, the injection chamber 110 forms a space, in which a sample is filled, together with the top surface of the lower substrate 200 on which a microgrid pattern 210 is established. The height of the injection chamber 110 can be adjusted appropriately according to the volume of the sample to be examined.

Preferably, the injection chamber 110 is formed 5 to 500 μm in height and, most preferably, 100 μm in height.

The sample inlet 120 is a portion through which a sample including microparticles is injected, and the outlet 130 is a portion through which air and an excess of the sample in the injection chamber 110 are discharged during the injection of the sample.

In a case where the sample inlet 120 and the outlet 130 are connected to the opposite sides in the injection chamber 110 of the upper substrate 100, it is easy to inject the sample into the injection chamber 110. Here, the outlet 130 acts as a vent hole through which the air is discharged during the injection of the sample. Accordingly, as the air in the injection chamber 110 is discharged through the outlet 130, the injection of the sample is made smoothly.

Meanwhile, a solvent channel 150 is formed along the circumference of the injection chamber 110 in the plastic microchip 10a of the present invention. Solvent inlets 140 including a plurality of openings connected to the solvent channel 150 are formed on the top surface of the upper substrate 100 so that the inner space of the solvent channel 150 is opened upward.

The solvent channel 150 is formed in a groove structure having predetermined height and width along the circumference of the injection chamber 110 on the bottom surface of the upper substrate 100. In a state where the upper substrate 100 is stacked on the lower substrate 200, the groove structure and the top surface of the lower substrate 200 form the solvent channel 150.

The solvent channel 150 is formed spaced apart from the circumference of the injection chamber 110 at regular intervals (directed to the thickness of a wall) along the whole circumference of the injection chamber 110, thus forming a wall 160.

Here, the inner surface of the solvent channel 150 corresponding to the outer surface of the wall 160 is formed vertically to the top surface of the lower substrate 200.

The solvent inlets 140 are provided to inject solvent into the solvent channel 150 so as to fix the upper substrate 100 to the lower substrate 200. The solvent inlets 140 are formed spaced apart from each other at regular intervals along the solvent channel 150.

Each of the solvent inlets 140 is established to ensure a sufficient space so that a solvent injection inlet such as a pipette inlet or an injection needle of a solvent injection device may smoothly enter the inside of the solvent channel 150 in the inclined direction.

Here, it is desirable that the width of the solvent inlets 140 be more than 1 mm so that the pipette inlet or the injection needle may smoothly enter a lower corner portion of the solvent channel 150, i.e., the boundary between the outer surface of the wall 160 of the upper substrate 100 and the top surface of the lower substrate 200.

In order to join the upper substrate 100, on which the solvent inlets 140 and the solvent channel 150 are formed, to the lower substrate 200, the upper substrate 100 is stacked on the lower substrate 200 and then the solvent is injected into the lower corner portion of the solvent channel 150 through the respective solvent inlets 140. Here, the injected solvent flows along the corner portion by a capillary phenomenon and thereby spreads all through the solvent channel 150. As a result, the two substrates 100 and 200 are welded to each other as the solvent penetrates into the interface between the two substrates.

In a state where the solvent is injected as described above, the wall 160 prevents the solvent flowing in along the solvent channel 150 from being introduced into the injection chamber 110 and further prevents the sample injected through the sample inlet 120 into the injection chamber 110 from leaking out of the injection chamber 110.

Moreover, since the solvent channel 150 provides a space through which the solvent injected through the solvent inlets 140 passes, it is desirable that the height of the solvent channel 150 in a closed section, of which the top is closed, i.e., in a section other than the solvent inlet section, be more than 0.2 mm so as to make the solvent flow smoothly along the lower corner portion of the solvent channel 150.

Here, if a sufficient height of the solvent channel 150 is not ensured, the solvent may spread over the other peripheral portion than the corner portion to cause a contamination and not to make the solvent flow smoothly, which results in a defective, thereby lowering the productivity.

Referring to a preferred embodiment depicted in FIG. 6, the injection chamber 110 is formed in a groove structure of a rectangular parallelepiped shape on the bottom surface of the upper substrate 100 and the volume of the injection chamber 110 can be calculated from the area and the height (depth of the groove structure) of the injection chamber 110.

Moreover, the sample inlet 120 and the outlet 130 are formed penetrating the upper substrate 100 vertically on both sides of the injection chamber 110, and the solvent channel 150 of a rectangular path is formed adjacent to the wall 160 along the circumference of the injection chamber 110. Furthermore, six solvent inlets 140 in total are formed in predetermined sections of the solvent channel 150 and thereby the corresponding portions of the top surface of the solvent channel 150 are opened on the upper substrate 100.

Of course, the embodiment depicted in the figure is just an example of the present invention and the present invention is not limited to the depicted embodiment. That is, it is possible to variously modify the shape of the injection chamber 110, the solvent channel 150 and the solvent inlets 140, and to appropriately change the number and position of the solvent inlets 140.

Next, the lower substrate 200 has no difference in the overall shape and structure from the conventional one; however, it has a significant feature in that the microgrid pattern 210 is formed in a negative structure, not a positive structure of the conventional one, on the top surface thereof.

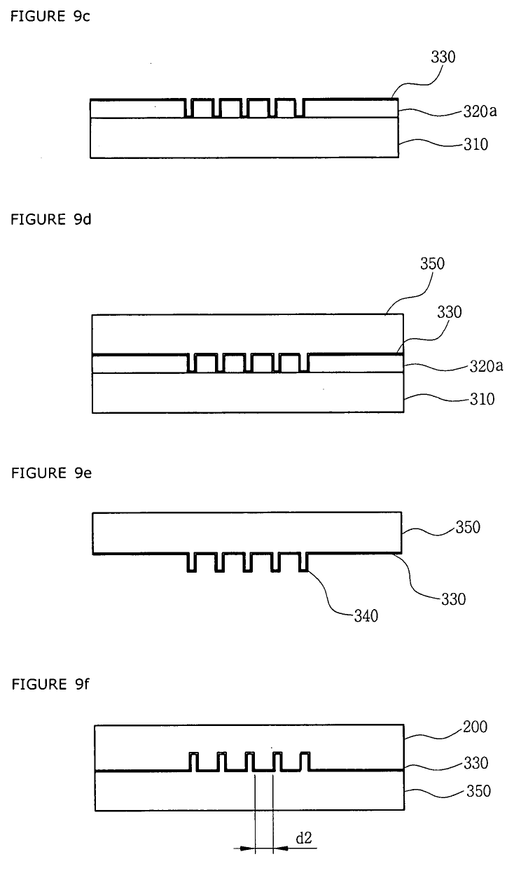

The negative microgrid pattern 210 is established in a predetermined region of the top surface of the lower substrate 200 including the region of the injection chamber 110, and the shape, depth (d4), width (d2) and interval (d3) thereof can be appropriately adjusted, if necessary (see FIGS. 9F and 9G).

Preferably, the respective grooves of horizontal and vertical microlines constituting the negative microgrid pattern 210 have a width of 4 μm or less and a depth of 1 μm or more. Moreover, the interval (d3) between the grooves is set larger than the width (d2) to be at least 5 μm.

Since a user should observe the microgrid pattern 210 in a smaller region to analyze a sample using analysis equipment such as a microscope, the more convenient it is to observe, the more narrow the interval (d3) of the microgrid pattern 210. Here, it is important that the width (d2) of the microgrid pattern should be set smaller in order to reduce the interval of the microgrid pattern 210.

Moreover, if the depth (d4) of the microgrid pattern 210 is formed deeply, it is possible to look at the microgrid pattern 210 clearly during the observation using the analysis equipment, thus facilitating the observation of sample.

Since the negative microgrid pattern 210, not the positive microgrid pattern of the conventional one, is formed in the plastic microchip 10a of the present invention, a clearer microgrid pattern 210 can be obtained during the injection molding of the lower substrate 200.

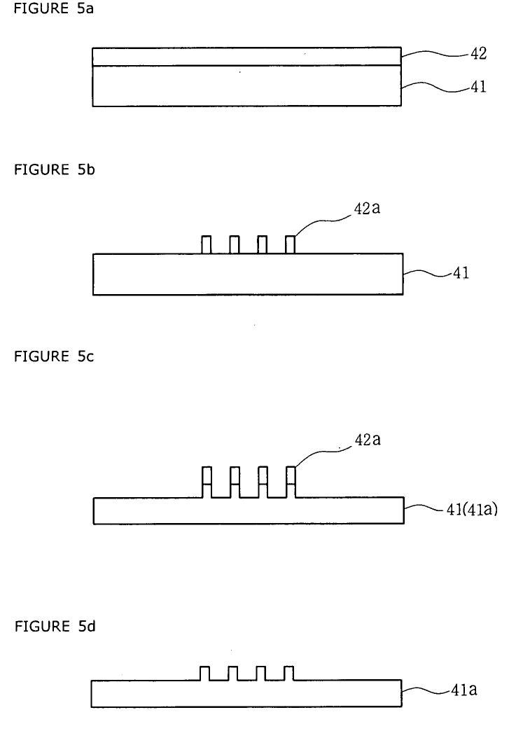

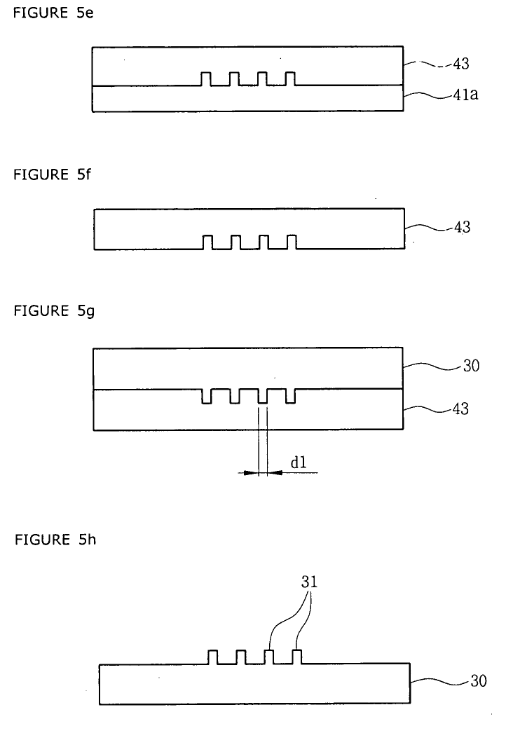

That is, in the positive microgrid pattern 31, as the molten resin is introduced into the narrow grooves formed on the mold 32, the pattern 31 protruding in a narrow width (d1) and in a predetermined height is formed on the surface of the lower substrate 30. That is, in order to form such a positive pattern 31, the molten resin should be injected into the narrow grooves of the mold (see FIGS. 4C, 4D, 5G and 5H). Accordingly, it is difficult to form a pattern 31 having a desired height, thus making the pattern unclear due to the unequal heights.

However, in the negative microgrid pattern 210, as the molten resin is smoothly introduced into a relatively wide space between the positive patterns formed by a stamper 350 to be described, the surface of the lower substrate 200 is formed. Subsequently, the grooves of the negative microgrid pattern 210 are formed on the surface of the lower substrate 200 by the positive pattern protruding on the surface of the stamper 350 (see FIGS. 9F and 9G). Accordingly, it is possible to obtain a clear negative pattern if the depths of the grooves are set deep.

The manufacturing process of the lower substrate 200 on which the negative microgrid pattern 210 is formed will be described later.

Meanwhile, the substrate region in which the injection chamber 110 is formed in the plastic microchip 10a should be formed transparently so as to observe the sample through the microscope. Accordingly, the upper substrate 100 and the lower substrate 200 are made of any light transmissive material.

Preferably, the upper substrate 100 and the lower substrate 200 are made by an injection molding process using any light transmissive plastic capable of injection molding such as polycarbonate (PC), polymethylmethacrylate (PMMA), polyethylene (PE), polypropylene (PP), polyethyleneterephthalate (PET), polystyrole (PS), cycloolefin (COC) resin, polyolefin (POC) resin, and so on. As the polyolefin resin, Zeon resin, Topas resin, etc. can be used.

Here, the light transmissiveness denotes that, when a light having a wavelength of 100 to 2,500 nm penetrates any material such as glass, plastic, etc., a transmittance of a specific region of the above wavelength band has 5% to 100%. The reason why such materials have the light transmissiveness is because the light of the above wavelength should penetrate the materials in order to facilitate analysis of microparticles such as cells, impurities, crystals, etc. with a naked eye or using analysis equipment.

The process of manufacturing the plastic microchip of the present invention will be described in detail as follows.

First, the upper substrate 100 depicted in FIGS. 6, 7A and 7B is formed.

Here, the configuration and materials of the upper substrate 100 are the same as described above and may be formed by an ordinary injection molding process.

Next, the lower substrate 200 depicted in FIGS. 6 and 8 is prepared.

Here, the materials of the lower substrate 200 and the microgrid pattern 210 formed in a negative structure are the same as described above and the lower substrate 200 may be formed by the injection molding process using a stamper 350 of a metal material on which a positive microgrid pattern 340 is formed.

An injection molding process used in an optical disk (CD) manufacturing process may be applied to the process of molding the lower substrate 200. The optical disk manufacturing process is directed to a method in which, after coating photoresist on a glass preform, its shape is moved onto a metal plate called the stamper through exposure, developing and plating processes, and the metal plate is mounted on a mold to obtain a plastic injection-molded product.

FIGS. 9A to 9G are sectional views illustrating respective processes of manufacturing the lower substrate 200 in accordance with the present invention. Referring to these figures, an example of the process of manufacturing the lower substrate 200 will be described in detail as follows.

As depicted in FIG. 9A, after preparing a plate 310 made of glass, silicon, ceramic, etc., a photoresist layer 320 is stacked on the plate 310 by coating photoresist (PR) using a spin coating process and the like.

Next, as depicted in FIG. 9B, the photoresist layer 320 is patterned through exposure and developing processes to form a mask pattern 320a including a negative microgrid pattern on the plate 310.

Subsequently, as depicted in FIG. 9C, a metal such as Cu, NI, etc. is stacked on the surface, on which the mask pattern 320a is formed, to be charged with electric current using a sputtering, vacuum deposition or electroless plating process, thus forming an electrically conductive metal layer 330.

Then, as depicted in FIG. 9D, a metal such as Cu, NI, etc. is stacked in a thickness of 0.1 mm or more on the metal layer 330 using an electroless plating or electroplating process, thus forming a stamper 350.

Here, if the metal is stacked in a thickness of 0.1 mm or less, it is difficult to mount the stamper 350 on a mold and thereby the injection molding process is not available.

Next, as depicted in FIG. 9E, after separating the plate and the mask pattern, the remaining photoresist is melted with an organic solvent or destroyed by fire to be removed, thus preparing a stamper 350 of a metal material.

The stamper 350 prepared in the form of a thin metal plate is used as a preform for injection molding the lower substrate 200.

Especially, the stamper 350 has a structure in which a positive microgrid pattern 340 is formed in a predetermined region. The positive microgrid pattern 340 used in molding a negative microgrid pattern 210 of a lower substrate 200 to be injection-molded later is formed in a position corresponding to the microgrid pattern 210 of the lower substrate 200.

Subsequently, the stamper 350 is finally completed by performing a series of processes of washing, coating a protective layer, polishing the rear side and cutting to a size capable of being fixed to a mold, and such processes are the same as the method used in the existing optical disk (CD) manufacturing process that is obvious to those skilled in the art.

It is desirable that the stamper 350 be made in a thickness of about 0.3 mm to ensure the lifespan and durability to be mounted on a mold.

Then, as depicted in FIG. 9F, the stamper 350 formed to have the positive microgrid pattern is mounted on the mold and then molten resin that is a material of the lower substrate 200 is injected by an injection molding device, thus forming a lower substrate 200 with a negative microgrid pattern.

Next, as depicted in FIG. 9G, if the mold is removed after the injection molding process, the lower substrate 200 including the negative microgrid pattern 210 is completed. Like this, the lower substrates 200 can be manufactured in a large quantity by repeating such an injection molding process.

In the case where the negative microgrid pattern 210 is formed on the lower substrate 200 using such a stamper 350 for the mass production, the molten plastic can be introduced into a relatively wide region to form the negative microgrid pattern 210 compared with the process of forming the positive microgrid pattern, that is, the molten plastic can be introduced more readily due to the difference between ‘d1’ of FIGS. 4C and 5G and ‘d2’ of FIG. 9F. Accordingly, it is possible to form a microgrid pattern of a relatively narrow width deeply and uniformly compared with the conventional one, thus obtaining a clear microgrid pattern 210.

Moreover, it is also possible to lower the temperature of the mold during the injection molding process considerably compared with the process of forming the positive microgrid pattern and thereby the cooling time can be reduced, thus increasing the productivity.

In manufacturing the lower substrate 200 as described above, the width (d3), depth (d4) and interval (d2) of the microgrid pattern 210 are the same as described above.

That is, as a preferred embodiment, the respective grooves of the microlines constituting the negative microgrid pattern 210 have a width of 4 μm or less and a depth of 1 μm or more. Moreover, the interval (d2) between the grooves is set larger than the width (d3) to be at least 5 μm.

Next, the upper substrate 100 and the lower substrate 200 manufactured as described above are fixed to each other to complete the plastic microchip 10a, and the fixing process will be described as follows.

It is desirable that the upper substrate 100 and the lower substrate 200 be formed in a body by welding the corresponding surfaces thereof to each other to be a stacked structure rather than using a method of using separate fixing means. Here, they may be welded to each other by an ordinary method, such as heating, using an adhesive, coating, pressurizing, vibrating, ultrasonic welding, etc. Preferably, a solvent welding process, in which a solvent, an adhesive or a mixture thereof is injected to a boundary between the two substrates through the solvent inlets 140 and the solvent channel 150, is used.

First, it is desirable that the surface treatment process for the upper substrate 100 and the lower substrate 200 be performed prior to the solvent welding process to increase the solvent flow rate. If the surface energy is increased through the surface treatment, the solvent flow rate is increased and thereby the connection state and force may be increased.

Moreover, if the upper substrate 100 and the lower substrate 200 are subjected to the surface treatment, the sample can smoothly flow along the flow path from the sample inlet 120, the injection chamber 110 to the outlet 130.

In the surface treatment process, the surfaces of the upper substrate 100 and the lower substrate 200 are subjected to hydrophilic and functional treatments using a surface treatment apparatus. Here, it is desirable to apply a surface modification process such as hydrophilic treatment, introduction of reactive groups, etc. using a plasma surface treatment apparatus that injects gas such as oxygen, nitrogen, argon, ammonia, etc. in a space under a low vacuum condition and, at the same time, applies a high voltage thereto.

If the plastic microchip 10a in accordance with the present invention is subjected to an oxygen plasma treatment so that it shows hydrophilic characteristics, an aqueous liquid such as blood can flow well in the injection chamber 110 and further spreads uniformly.

For example, in case of the hydrophilic treatment, the plastic microchip 10a is subjected to a plasma discharge treatment for 250 to 350 seconds by injecting oxygen gas of about 180 to 200 cm³/min.

Moreover, in order to introduce a desired reactive group, e.g. an amine group, it is possible to surface-treat the plastic microchip 10a by a plasma treatment with the amine-reactive group or by other chemical processes.

Like this, if the plastic microchip 10a is subjected to the surface treatment, it can be used in constituting a protein chip, a gene chip, etc., and their performance is improved more and more.

Meanwhile, to fix the upper substrate 100 and the lower substrate 200 subjected to the surface treatment as described above to each other, the two substrates 100 and 200 are stacked up and down and then the solvent is injected into the lower corner portion of the solvent channel 150, i.e., the boundary between the outer surface of the wall 160 of the upper substrate 100 and the top surface of the lower substrate 200, using a solvent injection device such as a pipette, an injection needle, etc. as described above.

Here, the solvent is injected through the respective solvent inlets 140 and the injected solvent flows from the outer surface of the wall 160 along the corner portion by a capillary phenomenon and thereby spreads all through the solvent channel 150.

As the solvent in the above-identified welding process, any organic solvent, adhesive or mixture thereof that can melt the materials of the upper substrate 100 and the lower substrate 200 may be used.

For example, it is possible to use at least one selected from the group consisting of ketones, aromatic hydrocarbons and halogenated hydrocarbons and, preferably, at least one selected from the group consisting of acetone, chloroform, methylene chloride and carbon tetrachloride is used.

Moreover, it is also possible to use a predetermined amount of adhesive such as acryl resin by mixing with the solvent or a mixture.

As described in detail above, if the solvent welding process is used in welding the upper substrate 100 and the lower substrate 200 to each other, it is possible to provide the height of the injection chamber 110 uniformly compared with the conventional process such as heating, using an adhesive, coating, pressurizing, vibrating, ultrasonic welding, etc.

Especially, if using the solvent welding process, it is possible to form the injection chambers 110 with a uniform height by the injection molding, since there is no change in the shape of the upper substrate 100 before and after the welding process compared with the convention ultrasonic welding process.

Like this, after the welding process of the upper substrate 100 and the lower substrate 200, a desired plastic microchip 10a is completed.

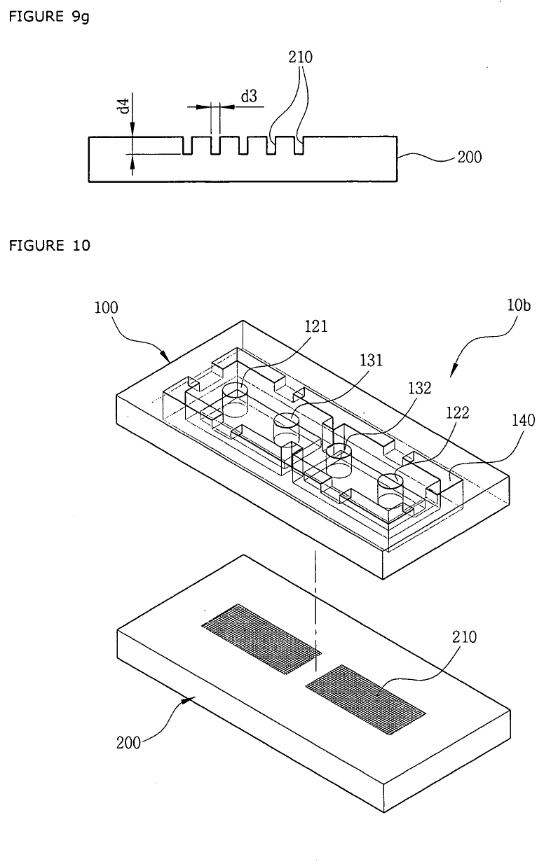

FIG. 10 is an exploded perspective view depicting another embodiment of the present invention including two injection chambers. As depicted in the figure, the plastic microchip 10b in accordance with another embodiment of the present invention includes two injection chambers, depicted in hidden lines divided by a wall. Each of the injection chambers includes a separate sample inlet 121 and 122 and an outlet 131 and 132.

Moreover, the solvent channels for the solvent welding, depicted in hidden lines, are formed along the circumference of the injection chambers in the upper substrate 100, and a plurality of solvent inlets 140 is formed at regular intervals along the respective solvent channels.

Like this, the plastic microchip 10b in accordance with the present invention can include more than two injection chamber, if necessary.

The plastic microchip of the present invention manufactured as described above can readily count the number of red blood cells, white blood cells, platelets, etc. contained in blood and cells contained in a sample such as spinal fluid, urine, saliva, milk etc. and further facilitate the counting and observation of mammalian germ cells.

Moreover, it is also possible to readily count the number of unicellular organisms such as bacteria, yeasts, etc., impurities contained in incompletely dissolved suspension, various metal and nonmetal crystals and any other microparticles.

As described above, the plastic microchip and the method for manufacturing the same provide the following advantages:

(1) It is possible to reduce the manufacturing time and cost, provide a mass production, and thereby to provide disposables at lower cost by injection molding a lower substrate on which a negative microgrid pattern is formed using a stamper for the mass production, compared with the conventional method in which a molten plastic material is put into a mold then cooled on the mold to be hardened;

(2) It is also possible to form a microgrid pattern of a relatively narrow width deeply and uniformly as a negative microgrid pattern is formed compared with the conventional one and thereby to obtain a clear microgrid pattern, thus facilitating accurate observation of microparticles using analysis equipment; and

(3) It is further possible to provide a uniform height of an injection chamber by welding an upper substrate and a lower substrate to each other by a solvent welding process, not affecting the surface properties of the injection chamber. Especially, since there is no change in the shape of the upper substrate before and after the welding process, it is thus possible to form the injection chambers with a uniform height by the injection molding, thus ensuring a more accurate analysis result.

As above, preferred embodiments of the present invention have been described and illustrated, however, the present invention is not limited thereto, rather, it should be understood that various modifications and variations of the present invention can be made thereto by those skilled in the art without departing from the spirit and the technical scope of the present invention as defined by the appended claims.

What is claimed is:

1. A plastic microchip for microparticle analysis comprising light transmissive upper and lower substrates stacked up and down, an injection chamber defined between the upper and lower substrates, a sample inlet connected to one side of the injection chamber, an outlet connected to the other side of the injection chamber, and a microgrid pattern formed on the top surface of the lower substrate, for counting the number of microparticles in a sample contained in the injection chamber,
wherein the microgrid pattern on the top surface of the lower substrate is formed in a negative structure in which microlines of a groove shape are arranged in the form of a lattice on the top surface of the lower substrate.

2. The plastic microchip for microparticle analysis as recited in claim 1,
wherein a groove structure is formed adjacent to a wall established along the whole circumference of the injection chamber on the bottom surface of the upper substrate, and the groove structure and the top surface of the lower substrate forming a solvent channel along a predetermined path of the circumference of the injection chamber, and
wherein a plurality of solvent inlets is formed penetrating the top surface of the solvent channel to be opened at regular intervals along the solvent channel on the upper substrate.

3. The plastic microchip for microparticle analysis as recited in claim 2,
wherein the width of the solvent inlets is made more than 1 mm and the height thereof is more than 0.2 mm.

4. The plastic microchip for microparticle analysis as recited in claim 2,
wherein the upper substrate and the lower substrate are welded by a solvent injected through the respective solvent inlets and then applied along a lower corner portion of the solvent channel including a boundary between the top surface of the lower substrate and the outer surface of the wall of the upper substrate.

5. The plastic microchip for microparticle analysis as recited in claim 1,
wherein respective grooves of horizontal and vertical microlines constituting the negative microgrid pattern have a width of 4 μm or less and a depth of 1 μm or more.

6. The plastic microchip for microparticle analysis as recited in claim 1,
wherein the upper substrate and the lower substrate are made of a light transmissive plastic having a transmittance of 5% to 100% for a light having a wavelength of 100 to 2,500 nm.

7. The plastic microchip for microparticle analysis as recited in claim 6,
wherein the upper substrate and the lower substrate are made of any one selected from the group consisting of polycarbonate (PC), polymethylmethacrylate (PMMA), polyethylene (PE), polypropylene (PP), polyethyleneterephthalate (PET), polystyrole (PS), cycloolefin (COC) resin and polyolefin (POC) resin.

8. A method for manufacturing a plastic microchip for microparticle analysis including light transmissive upper and lower substrates stacked up and down, an injection chamber formed between the upper and lower substrates, a sample inlet connected to one side of the injection chamber, an outlet connected the other side of the injection chamber, and a microgrid pattern, formed on the top surface of the lower substrate, for counting the number of microparticles in a sample of the injection chamber,
the method comprising the steps of:
(a) manufacturing an upper substrate by injection molding a light transmissive plastic;
(b) manufacturing a lower substrate including a negative microgrid pattern formed on the top thereof by injection molding a light transmissive plastic;
(c) surface-treating the upper substrate and the lower substrate; and
(d) welding the upper substrate and the lower substrate to be stacked up and down.

9. The method for manufacturing a plastic microchip for microparticle analysis as recited in claim 8,
wherein, in step (a), the upper substrate having a groove structure, formed adjacent to a wall provided along the whole circumference of the injection chamber on the bottom surface of the upper substrate, and a plurality of solvent inlets formed penetrating the top to be opened in the groove structure; and
wherein, in step (d), the upper substrate and the lower substrate stacked up and down are solvent-welded by injecting a solvent through the respective solvent inlets into a solvent channel formed by the groove structure and the top surface of the lower substrate, the solvent being injected into a boundary between the upper substrate and the lower substrate.

10. The method for manufacturing a plastic microchip for microparticle analysis as recited in claim 9,
wherein the solvent is applied along a lower corner portion of the solvent channel including the top surface of the lower substrate and the outer surface of the wall of the upper substrate.

11. The method for manufacturing a plastic microchip for microparticle analysis as recited in claim 9,
wherein the solvent is at least one selected from the group consisting of ketones, aromatic hydrocarbons, halogenated hydrocarbons, and a mixture thereof.

12. The method for manufacturing a plastic microchip for microparticle analysis as recited in claim 11,
wherein the solvent is at least one selected from the group consisting of acetone, chloroform, methylene chloride, carbon tetrachloride and a mixture thereof.

13. The method for manufacturing a plastic microchip for microparticle analysis as recited in claim 11,
wherein the solvent is a mixture to which an adhesive is added.

14. The method for manufacturing a plastic microchip for microparticle analysis as recited in claim 8,
wherein, step (b) comprises the steps of:
stacking a photoresist layer on a plate;
forming a mask pattern having a negative microgrid pattern on the plate by patterning the photoresist layer through exposure and developing processes;
forming an electrically conductive metal layer on the surface on which the mask pattern is formed;
forming a stamper of a metal material, on which a positive microgrid pattern is formed, on the metal layer by performing an electroless plating or electroplating;
separating the stamper from the mask pattern and washing the stamper separated;
processing the resulting stamper through a series of processes of coating a protective layer, polishing the rear side and cutting to a size capable of being fixed to a mold; and
obtaining a lower substrate on which a negative microgrid pattern is formed by mounting the processed stamper on the mold and then injection molding.

15. The method for manufacturing a plastic microchip for microparticle analysis as recited in claim 14,
wherein, in the step of forming a metal layer, Cu or Ni is deposited on the surface, on which the mask pattern is formed, by one selected from the group consisting of sputtering, vacuum deposition and electroless plating.

16. The method for manufacturing a plastic microchip for microparticle analysis as recited in claim 14,
wherein, in the step of forming a stamper, a stamper of a metal material of Cu or Ni having a thickness of 0.1 mm or more is formed by using an electroless plating or electroplating process.

17. The method for manufacturing a plastic microchip for microparticle analysis as recited in claim 12,
wherein the solvent is a mixture to which an adhesive is added.
